(19) United States
(12) Patent Application Publication
(10) Pub. No.: US 2026/0168013 A1
(43) Pub. Date: Jun. 18, 2026

(54) APPLICATION OF SACCHARIDE NANOMATERIAL IN DNA POLYMERASE

(71) Applicant: National Taiwan Ocean University, Keelung (TW)

(72) Inventors: Chih-Ching Huang, Keelung (TW); Ju-Yi Mao, Kaohsiung (TW); Yu Tang, Keelung (TW); Chen-Yu Lee, Keelung (TW); Chen-yow Wang, Yunlin County (TW)

(21) Appl. No.: 19/401,929

(22) Filed: Nov. 26, 2025

Related U.S. Application Data
(63) Continuation of application No. PCT/CN2024/139664, filed on Dec. 16, 2024.

(51) Int. Cl. C12Q 1/6848; C12N 9/99; C12Q 1/686

(52) U.S. Cl. CPC C12Q 1/6848; C12N 9/99; C12Q 1/686

(57) ABSTRACT

The present invention related to a saccharide nanomaterial for inhibiting DNA polymerase activity. The saccharide nanomaterial is produced by dry heating of saccharides and comprises graphene-like nanosheets and the saccharide. The graphene-like nanosheets contain at least a portion of the carbonized product of the saccharide, and form a cross-linked supramolecular structure with the saccharide. The saccharide nanomaterial can be applied to hot-start polymerase chain reaction (PCR), improving the sensitivity and specificity of the reaction.

### CROSS-REFERENCE TO RELATED PATENT APPLICATION(S)

This application is a continuation application of International Application No. PCT/CN2024/139664, filed on Dec. 16, 2024, the disclosures of which are hereby incorporated by reference.

### BACKGROUND OF THE INVENTION

### 1. Field of the Invention

The present invention relates to nanotechnology engineering and polymerase chain reactions, and in particular to saccharide nanomaterials and their use in inhibiting DNA polymerase.

### 2. Description of the Prior Art

Polymerase chain reaction (PCR) is a commonly employed technique in molecular biology for amplifying a specific nucleic acid sequence. The technique utilizes DNA polymerase to replicate nucleic acid sequences in vitro so as to amplify a target nucleic acid fragment by more than a million-fold, thereby increasing a minute sample to an amount sufficient for analysis. The technique can be applied to qualitative and quantitative analysis, sequencing, and detection of nucleic acids, and its fields of application are extensive.

Hot-start polymerase chain reaction (HS-PCR) is an improvement over conventional PCR designed to enhance amplification specificity and sensitivity. In HS-PCR, the activity of the DNA polymerase is controlled to reduce non-specific amplification such as primer-dimer formation. Before the reaction mixture is heated to the optimal working temperature, the DNA polymerase may initiate undesired amplification at lower temperatures, resulting in non-specific products that compromise accuracy. In hot-start PCR, the activity of the DNA polymerase is inhibited prior to initiating the reaction, thereby preventing non-specific amplification during the setup phase, and the polymerase activity is restored only after the PCR temperature rises to a defined level.

Current hot-start PCR approaches commonly employ antibodies or aptamers as ligands to inhibit polymerase activity. Such ligands bind to the active site of the DNA polymerase to suppress its activity at low temperature. When the PCR mixture is heated to the denaturation temperature, the antibody or aptamer loses activity and releases the DNA polymerase, thereby restoring polymerase function for amplification of the target sequence. However, because antibodies and aptamers inhibit effectively only at low temperature and are highly unstable during thermal cycling, they cannot maintain inhibition throughout temperature elevation and undergo denaturation at high temperature, precluding reuse during the reaction. In addition, their preparation is relatively complex and costly. Accordingly, there remains a need in the art for DNA polymerase-inhibitory materials that are more stable and possess reversible characteristics.

### SUMMARY OF THE INVENTION

In view of the foregoing, the present invention relates to a carbonized nanomaterial exhibiting DNA polymerase-inhibitory activity. The material achieves suppression of polymerase activity by binding to DNA polymerase and, in response to temperature changes, confers reversible inhibition. As a result, the carbonized nanomaterial effectively improves the specificity and sensitivity of PCR reactions. In addition, it is easy to prepare and cost-effective.

In one aspect, the present invention provides a method for inhibiting the activity of DNA polymerase, comprising adding a saccharide nanomaterial to a DNA polymerase solution, wherein the saccharide nanomaterial is produced by dry heating a saccharide at 150° C. to 300° C., wherein the saccharide nanomaterial comprises graphene-like nanosheet and the saccharide, wherein the graphene-like nanosheet comprise a carbonized product of at least a portion of the saccharide, and the graphene-like nanosheet is complexed with the saccharide to form a cross-linked supramolecular structure.

In some embodiments, the saccharide is selected from the group consisting of monosaccharide, disaccharide, oligosaccharide, polysaccharide and any combination thereof.

In some embodiments, the saccharide is selected from the group consisting of glucose, maltose, dextrin, sodium alginate, agarose, hydroxyethyl cellulose, and xanthan gum.

In some embodiments, the cross-linked supramolecular structure further has a functional group selected from the group consisting of hydroxyl, ester, phenol, carboxyl and any combination thereof on a surface thereof.

In some embodiments, the saccharide nanomaterial inhibits the activity of the DNA polymerase by binding to the DNA polymerase, and the inhibition is reversible.

In some embodiments, the method is used in a hot-start polymerase chain reaction, wherein the saccharide nanomaterial binds to the DNA polymerase to inhibit its activity, and the DNA polymerase is released from the saccharide nanomaterial upon an increase in reaction temperature, thereby restoring the activity of DNA polymerase.

In another aspect, the present invention provides a method for improving the accuracy of polymerase chain reactions, comprising adding a saccharide nanomaterial to the polymerase chain reaction, wherein the saccharide nanomaterial is produced by dry heating a saccharide at 150° C. to 300° C., wherein the saccharide nanomaterial comprises graphene-like nanosheet and the saccharide, wherein the graphene-like nanosheet comprise a carbonized product of at least a portion of the saccharide, and the graphene-like nanosheet is complexed with the saccharide to form a cross-linked supramolecular structure.

In some embodiments, the saccharide nanomaterial binds to the DNA polymerase to inhibit its activity, and the DNA polymerase is released from the saccharide nanomaterial upon an increase in reaction temperature, thereby restoring the activity of DNA polymerase.

In some embodiments, the concentration of the saccharide nanomaterial is 1 μg/ml to 2500 μg/ml.

In some embodiments, the polymerase chain reaction comprises conventional polymerase chain reaction (PCR), hot-start polymerase chain reaction (hot-start PCR), capillary electrophoresis polymerase chain reaction (PCR-capillary electrophoresis), real-time polymerase chain reaction (real-time PCR), multiplex polymerase chain reaction (multiplex PCR), or allele-specific polymerase chain reaction (allele-specific PCR).

The saccharide nanomaterial of the present invention is produced by an environmentally friendly, easily scalable, and highly reproducible one-pot synthesis. The process employs cost-effective and readily available saccharides or saccharide precursors, and does not require any catalyst or toxic solvent.

The present invention utilizes a carbonization-based synthesis in which saccharides are subjected to optimized conditions of thermal conversion temperature, reaction time, and purification so that, upon carbonization, the resulting product-depending on the choice of precursor-exhibits distinct carbonized structural morphologies and distributions of surface functional groups. The saccharide nanomaterial provided herein possesses reversible DNA polymerase-inhibitory properties and, by virtue of enhanced thermal stability under PCR conditions, imparts a hot-start function. Accordingly, it mitigates multiple banding and primer-dimer formation, lowers the threshold cycle (Ct) in qPCR, increases PCR amplification yield, improves multiplex-PCR efficiency, enhances variant-detection capability, and affords more specific genotyping results, thereby achieving higher analytical accuracy.

### BRIEF DESCRIPTION OF THE DRAWINGS

FIG. 1 is a schematic illustration of the generation of the saccharide nanomaterial of the present invention.

FIG. 2 shows the inhibitory effect of saccharide nanomaterials derived from different saccharide precursors on DNA polymerase.

FIG. 3A shows photographs of sodium alginate after dry heating at 150° C., 200° C., 250° C., and 300° C. (upper images) and after dissolution in deionized water (lower images). FIG. 3B shows transmission electron microscopy (TEM) images of sodium alginate after dry heating at 150° C., 200° C., 250° C., and 300° C.; the scale bars are 1000 nm, 2000 nm, 250 nm, and 500 nm, respectively. FIG. 3C shows high-resolution transmission electron microscopy (HRTEM) images of sodium alginate after dry heating at 150° C., 200° C., 250° C., and 300° C.; the scale bar is 5 nm, and the length of the marker is 0.24 nm.

FIG. 4 shows Fourier transform infrared (FT-IR) spectra of saccharide nanomaterials generated from sodium alginate after dry heating at 150° C., 200° C., 250° C., and 300° C., with a table correlating absorption peaks with corresponding functional groups.

FIG. 5A shows the electrophoresis result of PCR experiments by adding saccharide nanomaterials generated from sodium alginate after dry heating at 150° C., 200° C., 250° C., and 300° C.; the size of target sequence is 653 bp. FIG. 5B shows the electrophoresis result of PCR experiments by adding saccharide nanomaterials generated from glucose after dry heating at 150° C., 180° C., and 210° C.; the size of target sequence is 406 bp; wherein the symbol “ [symbol] ” indicates the position of the target sequence.

FIG. 6 shows the effect of saccharide nanomaterials generated from sodium alginate after dry heating at 250° C. on the relative activity of DNA polymerase at 35° C., 45° C., 55° C., 65° C., and 75° C. (*** indicates p<0.001).

FIG. 7A shows electrophoresis results illustrating the effect of saccharide nanomaterials generated from sodium alginate after dry heating at 250° C. on PCR at different DNA template concentrations; lanes 1, 2, 3, 4, and 5 correspond to 30, 3, 0.3, 0.03, and 0 ng DNA template, respectively. FIG. 7B shows electrophoresis results for PCR reactions adding 0 μg/mL, 0.01 μg/mL, 0.1 μg/mL, 1 μg/mL, 10 μg/mL, and 100 μg/mL of the saccharide nanomaterials generated from sodium alginate after dry heating at 250° C. FIG. 7C shows electrophoresis results of PCR products with target sizes of 280 bp, 320 bp, 406 bp, 653 bp, and 941 bp, obtained without (left) and with (right) the addition of the saccharide nanomaterial of the present invention. FIG. 7D shows electrophoresis results comparing the saccharide nanomaterial of the present invention (lane 1) with commercial hot-start PCR reagents, including DreamTaq DNA polymerase (Thermo Scientific, USA) (lane 2), DreamTaq Hot Start DNA polymerase (Thermo Scientific, USA) (lane 3), and KAPA2G Fast HotStart PCR Kit (Roche, Switzerland) (lane 4); wherein the symbol “ [symbol] ” indicates the position of the target sequence. FIG. 7E shows electrophoresis results for commercial PCR reagent kits operated without (control, upper image) and with (lower image) addition of the saccharide nanomaterial of the present invention, wherein the symbol “ [symbol] ” indicates the position of the target sequence.

FIG. 8A shows a comparative result of PCR detection of SARS-CoV-2 samples with addition of the saccharide nanomaterial of the present invention (** indicates p<0.01; *** indicates p<0.001; n=3). FIG. 8B shows a comparative result of PCR detection of SARS-CoV-2 mutant samples of the omicron variant targeting the envelope (E) gene with addition of the saccharide nanomaterial (* indicates p<0.05; n=3). FIG. 8C shows a comparative result of primer-dimer levels in PCR detection of ALDH2 (E487K) SNP mutant samples with addition of the saccharide nanomaterial (* indicates p<0.05; n=3).

FIG. 9A shows real-time PCR amplification curves (left) and melting-curve (right) for samples at a virus concentration of 2000 PFU with or without addition of the saccharide nanomaterial. FIG. 9B shows real-time PCR amplification curves (left) and melting-curve (right) for samples at a virus concentration of 200 PFU with or without addition of the saccharide nanomaterial. FIG. 9C shows analysis of Ct values from real-time PCR detection across samples with different virus concentrations.

### DETAILED DESCRIPTION OF THE PREFERRED EMBODIMENT

The following examples are provided to illustrate the embodiments of the present disclosure. People skilled in the art will readily understand the advantages and efficacy of the present disclosure based on the teachings revealed in this specification. The present disclosure may also be implemented or applied in other different embodiments. The details provided in this specification may be modified and altered from various perspectives and applications without departing from the scope disclosed herein.

Unless otherwise defined herein, scientific and technical terms used in connection with the present disclosure shall have the meanings that are commonly understood by those of ordinary skill in the art. The terminology used in this specification is used to describe particular embodiments only and is not intended to limit the invention.

As used herein, the singular form “a”, “an”, and “the” includes plural references unless indicated otherwise. For example, “an” excipient includes one or more excipients. The term “or” is used interchangeably with the term “and/or” unless the context clearly indicates otherwise.

As used interchangeably herein, “around”, “about” and “approximately” shall generally mean plus or minus 5% of the numerical value of the number with which it is being used. Numerical quantities given herein are approximate, meaning that the term “around”, “about” or “approximately” can be inferred if not expressly stated.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

In one aspect, the present invention provides a method for inhibiting the activity of DNA polymerase, comprising adding a saccharide nanomaterial to a DNA polymerase solution, wherein the saccharide nanomaterial is produced by dry heating a saccharide at 150° C. to 300° C., wherein the saccharide nanomaterial comprises graphene-like nanosheet and the saccharide, wherein the graphene-like nanosheet comprise a carbonized product of at least a portion of the saccharide, and the graphene-like nanosheet is complexed with the saccharide to form a cross-linked supramolecular structure.

In at least one embodiment, dry heating is carried out at temperatures between 150° C. and 300° C., for example, with a lower limit of 150° C. or an upper limit of 300° C. In some embodiments, the heating temperature is in the range of 180° C. to 300° C., for example, approximately 190° C., approximately 200° C., approximately 210° C., approximately 220° C., approximately 230° C., approximately 240° C., approximately 250° C., approximately 260° C., approximately 270° C., approximately 280° C., and approximately 290° C.

In some embodiments, the saccharide is selected from the group consisting of monosaccharide, disaccharide, oligosaccharide, polysaccharide and any combination thereof. In some embodiments, the saccharide comprises the saccharide precursor, wherein the saccharide precursor is selected from the group consisting of hydroxyl and carboxyl groups. In some embodiments, the saccharide is selected from the group consisting of tetracarbon sugar monomers, tetracarbon sugar polymers, pentose sugar monomers, and pentose sugar polymers.

In some embodiments, the saccharide is selected from the group consisting of glucose, maltose, dextrin, sodium alginate, agarose, hydroxyethyl cellulose, and xanthan gum.

In some embodiments, the cross-linked supramolecular structure further has a functional group selected from the group consisting of hydroxyl, ester, phenol, carboxyl and any combination thereof on a surface thereof. The saccharide nanomaterials described in present invention are obtained through the condensation of sugar ring opening and closing and the reaction regulation of benzene ring cyclization, resulting in a complex cross-linked supramolecular structure with polycyclic aromatic phenols.

In some embodiments, the saccharide nanomaterial inhibits the activity of the DNA polymerase by binding to the active site of DNA polymerase through its functional groups, and the inhibition effect is reversible.

In some embodiments, the method is used in a hot-start polymerase chain reaction, wherein the saccharide nanomaterial binds to the DNA polymerase to inhibit its activity, and the DNA polymerase is released from the saccharide nanomaterial upon an increase in reaction temperature, thereby restoring the activity of DNA polymerase. The inhibition of DNA polymerase activity by the saccharide nanomaterial is reversible. As the temperature changes, it affects the binding or release of DNA polymerase by the saccharide nanomaterial. Therefore, the inhibitory activity can be changed by regulating the temperature.

In another aspect, the present invention provides a method for improving the accuracy of polymerase chain reactions, comprising adding a saccharide nanomaterial to the polymerase chain reaction, wherein the saccharide nanomaterial is produced by dry heating a saccharide at 150° C. to 300° C., wherein the saccharide nanomaterial comprises graphene-like nanosheet and the saccharide, wherein the graphene-like nanosheet comprise a carbonized product of at least a portion of the saccharide, and the graphene-like nanosheet is complexed with the saccharide to form a cross-linked supramolecular structure. This accuracy includes both sensitivity and specificity.

In some embodiments, the saccharide nanomaterial binds to the DNA polymerase to inhibit its activity, and the DNA polymerase is released from the saccharide nanomaterial upon an increase in reaction temperature, thereby restoring the activity of DNA polymerase. The saccharide nanomaterial further endows the polymerase chain reaction with hot-start properties during temperature changes.

In some embodiments, the concentration of the saccharide nanomaterial is 1 μg/ml to 2500 μg/ml. For example, 3 μg/ml, 5 μg/ml, 10 μg/ml, 15 μg/ml, 20 μg/ml, 25 μg/ml, 30 μg/ml, 35 μg/ml, 40 μg/ml, 45 μg/ml, 50 μg/ml, 55 μg/ml, 60 μg/ml, 65 μg/ml, 70 μg/ml, 75 μg/ml, 80 μg/ml, 85 μg/ml, 90 μg/ml, 95 μg/ml, 100 μg/ml, 150 μg/ml-200 μg/m, 250 μg/ml, 300 μg/ml, 350 μg/ml, 400 μg/ml, 450 μg/ml, 500 μg/ml, 550 μg/m, 600 μg/ml, 650 μg/ml, 700 μg/ml, 750 μg/ml, 800 μg/ml, 850 μg/ml, 900 μg/m, 950 μg/ml, 1000 μg/ml, 1100 μg/ml, 1200 μg/ml, 1300 μg/ml, 1400 μg/ml, 1500 μg/ml, 1600 μg/ml, 1700 μg/ml, 1800 μg/ml, 1900 μg/ml, 2000 μg/ml, 2200 μg/ml, 2250 μg/ml, 2300 μg/ml, 2350 μg/ml, 2400 μg/ml, 2450 μg/ml and 2500 μg/ml

In some embodiments, the polymerase chain reaction comprises conventional polymerase chain reaction (PCR), hot-start polymerase chain reaction (hot-start PCR), capillary electrophoresis polymerase chain reaction (PCR-capillary electrophoresis), real-time polymerase chain reaction (real-time PCR), multiplex polymerase chain reaction (multiplex PCR), or allele-specific polymerase chain reaction (allele-specific PCR).

As used herein, the term “saccharide nanomaterial” refers to a carbonized product obtained by the dry-heating of saccharides as disclosed herein. Under elevated temperatures, saccharide precursors undergo decomposition and rearrangement to form carbon-based nanomaterials. In bottom-up thermal synthesis of carbon quantum dots (CQDs) and carbonized polymer dots (CPDs), domains bearing various functional groups are generated during the process and become embedded on the surface or within the structure of the material; such functional groups confer specific chemical or physical properties to the material.

As used herein, “polymerase chain reaction (PCR)” requires at least a template DNA, a forward primer, a reverse primer, deoxynucleoside triphosphates (dNTPs), a Taq DNA polymerase, and a buffer solution. As used herein, “hot-start polymerase chain reaction” refers to a PCR format in which the activity of the DNA polymerase is temporarily suppressed at the outset of the reaction to prevent nonspecific amplification and primer-dimer formation at lower temperatures, and the enzyme is allowed to regain activity only after the reaction temperature has been raised to a defined level, thereby improving the accuracy of amplification. As used herein, “hot start” generically denotes the process by which a DNA polymerase becomes activated upon elevation of temperature.

As used herein, “saccharide” is a generic term for aldehydes or ketones bearing at least two hydroxyl groups and their condensation products, or derivatives thereof, composed primarily of carbon, hydrogen, and oxygen, comprising monosaccharides, disaccharides, oligosaccharides, and polysaccharides. As used herein, “saccharide derivative” refers to a compound obtained by chemical reaction or modification of a saccharide, which generally retains structural features of the original saccharide while undergoing chemical change at one or more positions (e.g., substitution, oxidation, reduction, or conjugation with other groups). For example, derivatives include sorbitol (reduced from glucose), gluconic acid (oxidized from glucose), and glucosamine (in which the hydroxyl group at the C2 position of glucose is replaced by an amino group). As used herein, “saccharide precursor” refers to a saccharide employed in the dry-heating process of the present invention to prepare the nanomaterial.

As used herein, “reversible” means that the association of substances can be disengaged upon changes in external conditions without permanently altering their structure or function; that is, the association between substances can be reversed. For example, the saccharide nanomaterial of the present invention may bind a DNA polymerase and release the DNA polymerase upon an increase in temperature, without altering the enzyme's structure or activity.

As used herein, “accuracy” refers to the ability of each reaction to faithfully amplify the target sequence fragment while avoiding amplification of non-target fragments or the formation of nonspecific products. As used herein, “improving the accuracy of polymerase chain reaction” refers to improving the sensitivity and specificity of the PCR, increasing amplification of the target fragment, and reducing the generation of nonspecific products.

In order to facilitate the understanding of the technical features, the contents, and the advantages of the present disclosure, and the effectiveness thereof that can be achieved, the present disclosure will be illustrated in detail below through embodiments with reference to the accompanying drawings.

### Example 1 Preparation Method of Saccharide Nanomaterials

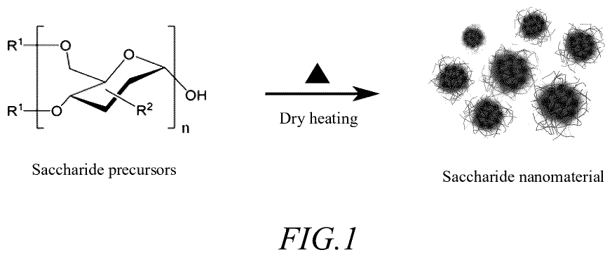

In this embodiment, a saccharide nanomaterial embedded with graphene-like nanosheets is synthesized by heating a saccharide precursor. As shown in FIG. 1, upon dry heating, the saccharide precursor undergoes ring-opening hydration, followed by polymerization and crosslinking, and subsequently condensation and aromatization, thereby yielding the saccharide nanomaterial of the present invention.

In this embodiment, 50 mg of a saccharide precursor was placed in a 20 mL glass sample vial and dry-heated for 3 hours at 150° C., 200° C., 250° C., or 300° C. in a laboratory-grade convection oven (DH 300, Dengyng, Taiwan). The resulting solid product was allowed to cool and then dissolved in 5.0 mL of deionized water by ultrasonic treatment for 1 hour. Larger particulates were removed by centrifugation at a relative centrifugal force (RCF) of 500×g for 30 minutes. The resulting saccharide nanomaterial dispersion was stored at 4° C. for later use.

In this embodiment, glucose, maltose, dextrin, sodium alginate, agarose, hydroxyethyl cellulose, and xanthan gum were respectively employed as saccharide precursors, and saccharide nanomaterials were prepared at 150° C., 200° C., 250° C., or 300° C. The yield after thermal decomposition (pyrolysis) is summarized in Table 1, and the yield of the soluble carbonized product (carbonized supernatant) obtained after centrifugal purification is summarized in Table 2.

TABLE 1
Yields of pyrolyzed products from different saccharide
precursor at different temperatures

| | |
|---|---|
| saccharide | Yield after dry pyrolysis (%) |

| precursor | 150° C. | 200° C. | 250° C. | 300° C. |
|---|---|---|---|---|
| glucose | >99 | 89 | 70 | 33 |
| maltose | >99 | 94 | 64 | 36 |
| dextrin | >99 | 98 | 70 | 25 |
| sodium alginate | >99 | 91 | 69 | 62 |
| agarose | >99 | >99 | 59 | 31 |
| hydroxyethyl | >99 | 91 | 61 | 41 |
| cellulose | | | | |
| xanthan gum | >99 | 94 | 61 | 49 |

TABLE 2
Yield of highly soluble carbonized products in supernatants
after purification by centrifugation.

| | |
|---|---|
| saccharide | Yield of carbonized supernatant (%) |

| precursor | 150° C. | 200° C. | 250° C. | 300° C. |
|---|---|---|---|---|
| glucose | >99 | 66 | <1 | <1 |
| maltose | >99 | 75 | 5 | <1 |
| dextrin | 94 | 93 | <1 | <1 |
| sodium alginate | 88 | 67 | 57 | 46 |
| agarose | 85 | 52 | <1 | <1 |
| hydroxyethyl | 81 | 38 | <1 | <1 |
| cellulose | | | | |
| xanthan gum | 96 | 22 | 18 | <1 |

### Example 2 Effects of DNA Polymerase Inhibition

In this embodiment, saccharide nanomaterials prepared from different saccharides and at different heating temperatures were co-incubated with a DNA polymerase during a polymerization reaction to assess the effect of the saccharide nanomaterials on DNA polymerase activity.

### Materials and Methods

The preparation method of the saccharide nanomaterial of this embodiment was performed as described in Example 1.

A 49-mer hairpin DNA was used as the substrate and template for a polymerization reaction catalyzed by DNA polymerase. When the polymerization proceeded normally using the 49-mer hairpin DNA as the template, a 64-mer hairpin DNA was generated. Conversely, when the DNA polymerase activity was inhibited, the 64-mer hairpin DNA was not produced. The DNA polymerase employed in this experiment was Taq DNA polymerase (Bio-Helix, Taiwan). The DNA polymerase activity assay was conducted at 35° C. for 80 minutes. Saccharide nanomaterials prepared from glucose (dry-heating temperatures: 150° C. and 200° C.), maltose (150° C. and 200° C.), dextrin (150° C. and 200° C.), sodium alginate (150° C., 200° C., 250° C., and 300° C.), agarose (150° C., 200° C., and 250° C.), hydroxyethyl cellulose (150° C. and 200° C.), and xanthan gum (150° C. and 200° C.) were tested separately. Upon completion of the reaction, the amounts of the two hairpin DNAs and their respective extension products were analyzed by capillary gel electrophoresis (CGE). DNA polymerase activity was quantified by calculating the peak-area ratio of the 64-mer product to the 49-mer substrate.

### Results

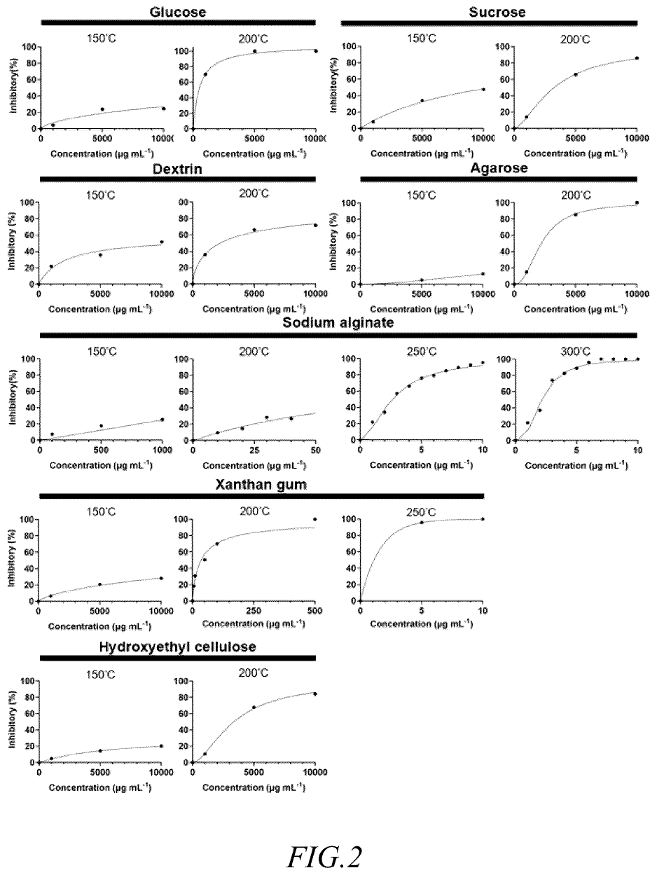

As shown in FIG. 2, saccharide nanomaterials prepared from different saccharide precursors at various heating temperatures exhibited concentration-dependent inhibition of DNA polymerase activity. Especially, sodium alginate dry-heated at 250° C. and 300° C. showed the strongest inhibitory effect.

### Example 3 Qualitative Analysis of Saccharide Nanomaterials

In this embodiment, sodium alginate was used as a saccharide precursor, and the effect of dry heating at 150° C., 200° C., 250° C., and 300° C. on the characteristics of the saccharide nanomaterial was investigated, so as to qualitatively characterize the saccharide nanomaterial described herein.

Alginates are composed of β-(1→4)-linked D-mannuronic acid (M) and α-(1→4)-linked L-guluronic acid (G) units. Sodium alginate is widely employed in the food industry, the pharmaceutical field, and numerous biomedical applications (e.g., wound dressings, drug delivery, and immunotherapy) due to its high biocompatibility, low cost, and gel-forming capacity upon addition of divalent cations (e.g., Mg²⁺ and Ca²⁺).

### Materials and Methods

The method for preparing the saccharide nanomaterial in this embodiment was as described in Example 1.

The particle size and morphology of the saccharide nanomaterial prepared from sodium alginate were analyzed using a transmission electron microscopy (TEM) system (Tecnai G2 F20 S-TWIN, Philips/FEI, Hillsboro, OR, USA) operated at 200 kV.

Binding energies were calibrated against the Cls peak at 284.6 eV Fourier transform infrared spectroscopy (FT-IR; FT/IR-6100, JASCO, Easton, MD, USA) was conducted in transmission mode over 500-4,000 cm⁻¹ with 16 scans to analyze functional groups that may be present in the sodium-alginate-derived nanomaterial.

### Results

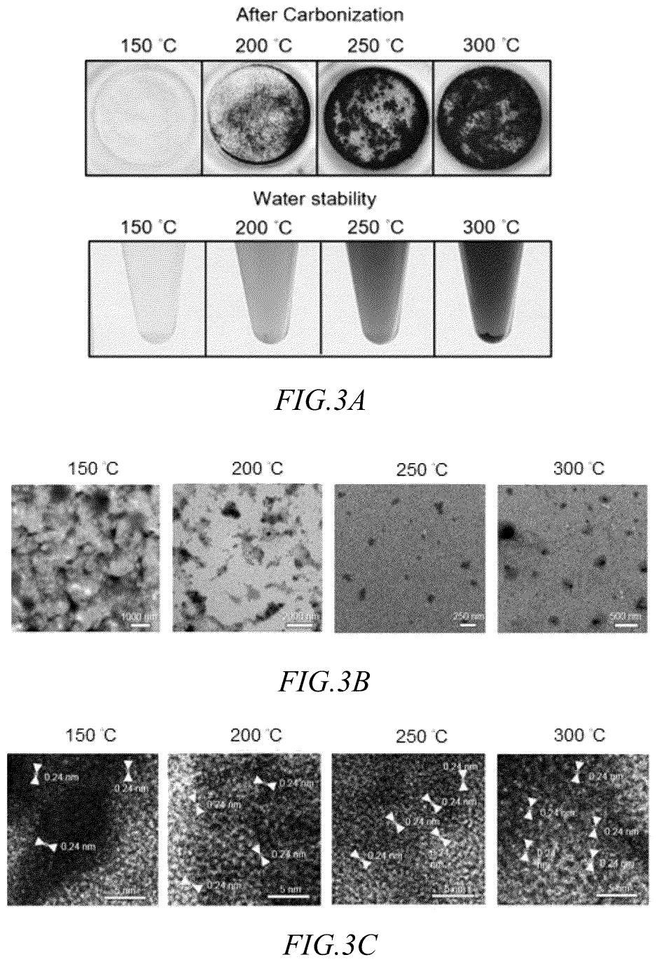

As shown in FIG. 3A, the upper panel shows the carbonized products obtained after dry heating sodium alginate at 150° C., 200° C., 250° C., and 300° C., and the lower panel shows the appearance after dissolution in deionized water followed by purification by centrifugation. As shown in FIG. 3B, at a heating temperature of 150° C., the sodium-alginate-derived nanomaterial contains gel-like cross-linked polymers formed via condensation reactions, whereas materials heated at 200-300° C. exhibit distinct particulates formed within a polymer matrix, indicating that the morphology of the saccharide nanomaterial is highly correlated with the heating temperature. As shown in FIG. 3C, sodium-alginate-derived nanomaterials heated to >200° C. display a finer polymer network together with discernible graphitic carbon layers having a d-spacing value of 0.24 nm, evidencing (100) and (112) lattice planes and revealing the formation of graphene-like crystalline structures in addition to the cross-linked nanogel matrix.

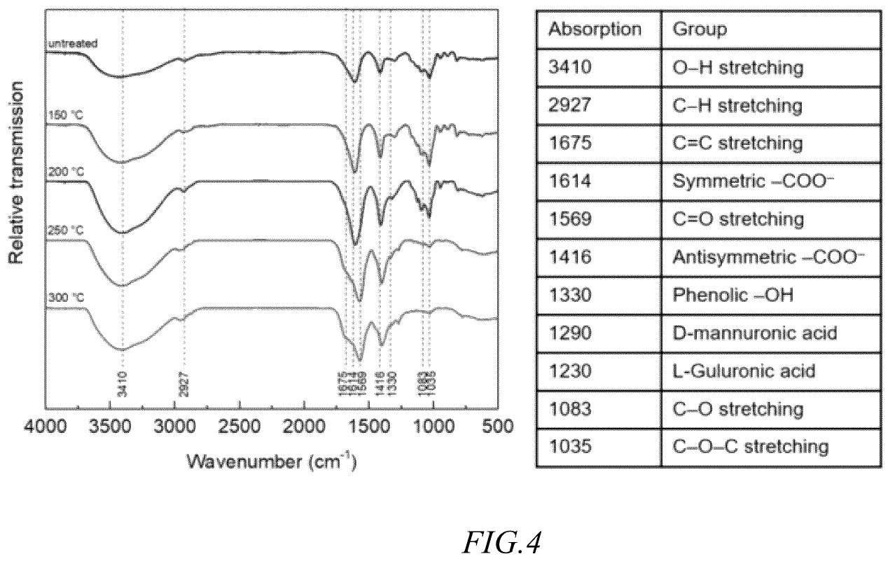

As shown in FIG. 4, FT-IR analysis demonstrates that, as the synthesis temperature increases from 200° C. to 300° C., the C—O stretching bands at 1035/1083 cm⁻¹ decrease markedly. In addition, the spectrum of the sodium-alginate-derived nanomaterial exhibits a band at 1330 cm⁻¹ attributable to 0-H bending of phenolic groups arising from mild aromatization during carbonization. Accordingly, the experimental results indicate that the saccharide nanomaterials described herein undergo mild carbonization that retains certain functional groups while generating new ones, and that the saccharide nanomaterials are enriched in specific functional groups, including, for example, hydroxyl, ester, phenolic, and carboxyl groups.

### Example 4 Effects of Polymerase Chain Reaction

In this embodiment, polymerase chain reaction (PCR) was performed using Taq DNA polymerase, and saccharide nanomaterials prepared at different temperatures were added to the reaction. The PCR products were subsequently visualized by gel electrophoresis imaging to assess the effect of the saccharide nanomaterial on PCR.

### 4.1 Sodium Alginate Nanomaterials

In this embodiment, sodium alginate was subjected to dry heating to form a sodium-alginate-derived nanomaterial, which was then added to a PCR reaction to analyze its effects.

### Materials and Methods

The method for preparing the saccharide nanomaterial in this embodiment was as described in Example 1.

PCR was performed using 30 ng of a 406 bp human gDNA template, with addition to the reaction of sodium-alginate-derived nanomaterials obtained by dry heating at 150° C., 200° C., 250° C., and 300° C. at final concentrations of 1, 10, and 100 μg/mL, followed by gel electrophoresis analysis. In the figure, “M” denotes a DNA ladder (Bio-Helix, Taiwan), “C” denotes a control without added sodium-alginate-derived nanomaterial, and the “unheated” group denotes addition of sodium alginate that had not undergone dry heating.

The PCR reaction was prepared by combining different concentrations of the saccharide nanomaterial with 2 μL of 10×PCR buffer (Bio-Helix, Taiwan), 200 M dNTPs, 5 U Taq DNA polymerase (Bio-Helix, Taiwan), a human genomic DNA template at 1,000 copies (Merck KGaA, Germany), and 500 nM forward and reverse primers to a final volume of 20 μL. Thermal cycling comprised 35 cycles, each including denaturation at 95° C. for 30 s, annealing at 54 or 64° C. for 30 s, and extension at 72° C. for 60 s. Amplified PCR products were separated by gel electrophoresis using 2% agarose and a DNA ladder (DM115-0100, Bio-Helix, Taiwan), stained with HealthView nucleic acid stain (Genomics, Taiwan), and visualized under ultraviolet illumination.

### Results

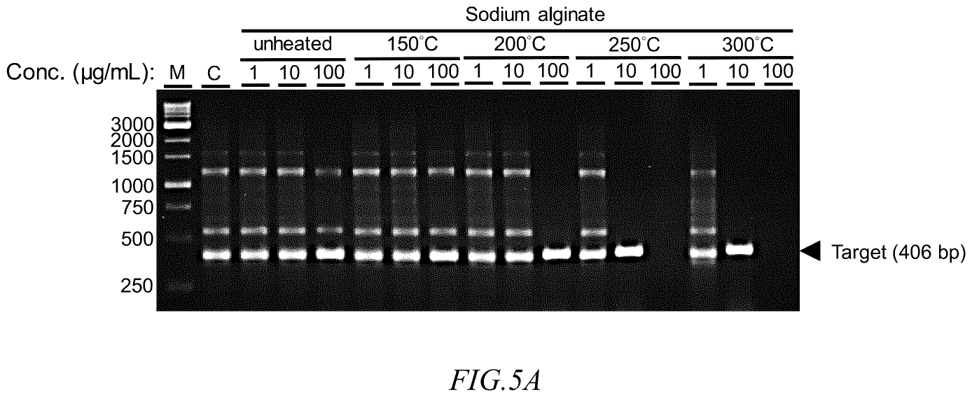

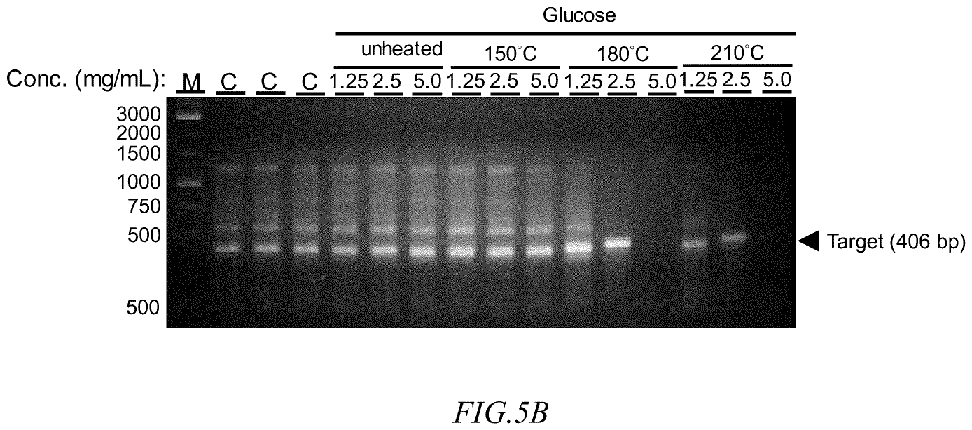

As shown in FIG. 5A, in the control (C) without sodium-alginate-derived nanomaterial, the PCR produced the correct 406-bp target amplicon together with numerous nonspecific products. This is attributable to the broad temperature range of DNA polymerase activity, which permits low-temperature extension and formation of nonspecific products. In contrast, upon addition of the sodium-alginate-derived nanomaterial, nonspecific products were reduced; notably, no nonspecific products were observed with the material prepared at 200° C. at 100 μg/mL, the material prepared at 250° C. at 10 μg/mL, or the material prepared at 300° C. at 10 μg/mL. Under these conditions, only robust, specific amplification of the 406-bp target fragment was evident, indicating substantially enhanced specificity and sensitivity of the DNA polymerase, similar to hot-start PCR employing antibody-inhibited polymerase. Additionally, the materials prepared at 250° C. and 300° C. exhibited stronger binding to Taq DNA polymerase and greater inhibitory effects; consequently, at a high concentration (100 μg/mL) no PCR amplification was observed.

### 4.2 Glucose Nanomaterials

In this embodiment, glucose was subjected to dry heating to form a glucose-derived nanomaterial, which was then added to a PCR reaction to evaluate its effects.

### Materials and Methods

The method for preparing the saccharide nanomaterial in this embodiment was as described in Example 1.

PCR was performed using 30 ng of a 406-bp human gDNA template. Glucose-derived nanomaterials obtained by dry heating at 150° C., 180° C., and 210° C. were added to the reaction at final concentrations of 1.25, 2.5, and 5 mg/mL, followed by gel electrophoresis. In the figure, “M” denotes a DNA ladder, “C” denotes a control without added glucose-derived nanomaterial (n=3), and the “unheated” group denotes addition of glucose that had not undergone dry heating. The PCR conditions were as described above.

### Results

As shown in FIG. 5B, in the control (C) without glucose-derived nanomaterial, the 406-bp target amplicon was not prominently observed and numerous nonspecific products were present. In contrast, upon addition of the glucose-derived nanomaterial prepared at 180° C. at 2.5 mg/mL or the material prepared at 210° C. at 2.5 mg/mL, nonspecific products disappeared and only a distinct target band was observed, indicating a marked increase in PCR specificity.

### Example 5 DNA Polymerase Inhibition Efficacy Under Different Conditions

In this embodiment, different concentrations of the saccharide nanomaterial were reacted with Taq DNA polymerase under different temperatures to determine the inhibitory effect of the saccharide nanomaterial on DNA polymerase as a function of concentration and temperature.

### Materials and Methods

The saccharide nanomaterial was prepared as described in Example 1, wherein sodium alginate was used as the saccharide precursor and the dry-heating temperature was 250° C.

The DNA polymerase activity assay was conducted as described in Example 2. Briefly, reactions containing DNA polymerase were incubated for 80 minutes at 35° C., 45° C., 55° C., 65° C., or 75° C. with 0 μg/mL, 2.5 μg/mL, 10 μg/mL, or 40 μg/mL of the saccharide nanomaterial. The group with 0 μg/mL saccharide nanomaterial (no addition) served as the control.

### Results

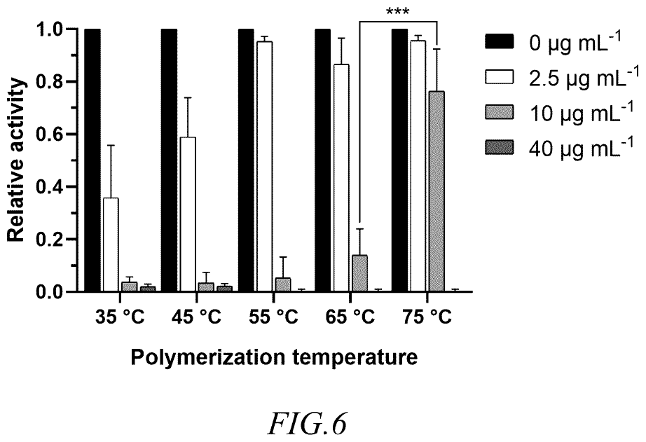

As shown in FIG. 6, the group added with 40 μg/mL of the sodium-alginate-derived nanomaterial exhibited obvious inhibition of DNA polymerase activity at all temperatures tested relative to the no-addition control. Notably, at 35° C., addition of 10 μg/mL of the sodium-alginate-derived nanomaterial effectively suppressed Taq DNA polymerase activity (>99%), and inhibition persisted at reaction temperatures of 45° C., 55° C., and 65° C. However, at 75° C., polymerase activity increased significantly compared with that at 65° C. and recovered to approximately 80% of the control level, indicating that at 75° C. Taq DNA polymerase is not inhibited by the sodium-alginate-derived nanomaterial. The 2.5 μg/mL group likewise showed a trend of increasing activity with increasing temperature. These results indicate that the saccharide nanomaterial effectively inhibits DNA polymerase activity at lower temperatures, whereas the inhibition is reversed at higher temperatures due to the release of the polymerase from the nanomaterial.

PCR primarily relies on Taq DNA polymerase to catalyze polymerization at temperatures >70° C. That is to say, the saccharide nanomaterial can effectively suppress polymerase activity during sample preparation and early amplification stages, thereby preventing nonspecific amplification. Furthermore, the results demonstrate a positive correlation between the concentration of the sodium-alginate-derived nanomaterial and the extent of DNA polymerase inhibition, enabling control of polymerase activity by adjusting the nanomaterial concentration.

### Example 6 the Efficacy of Hot-Start Polymerase Chain Reaction

In this embodiment, the saccharide nanomaterial was added to polymerase chain reactions (PCR) to evaluate its effects on specificity, sensitivity, and accuracy, and the results were compared with those obtained using commercially available hot-start PCR kits. In addition, the effects of the saccharide nanomaterial on different DNA polymerases were further investigated.

The saccharide nanomaterial was prepared as described in Example 1, wherein sodium alginate was used as the saccharide precursor and the dry-heating temperature was 250° C. Unless otherwise indicated, the PCR conditions were as set forth in Example 4, and the reaction conditions for each commercial PCR kit were conducted in accordance with the respective user manuals.

### 6.1 Concentration Test of DNA Template

This experiment evaluated the effects of the saccharide nanomaterial and varying template concentrations on PCR amplification performance.

### Materials and Methods

PCR was performed using human gDNA (500 bp target) at template inputs of 30 ng (lane 1), 3 ng (lane 2), 0.3 ng (lane 3), 0.03 ng (lane 4), and 0 ng (lane 5). The experimental group contained 10 μg/mL of the sodium-alginate-derived nanomaterial, and the control group contained no saccharide nanomaterial. The PCR products were subsequently analyzed by gel electrophoresis. Unless otherwise indicated, PCR conditions were as set forth in Example 4.

### Results

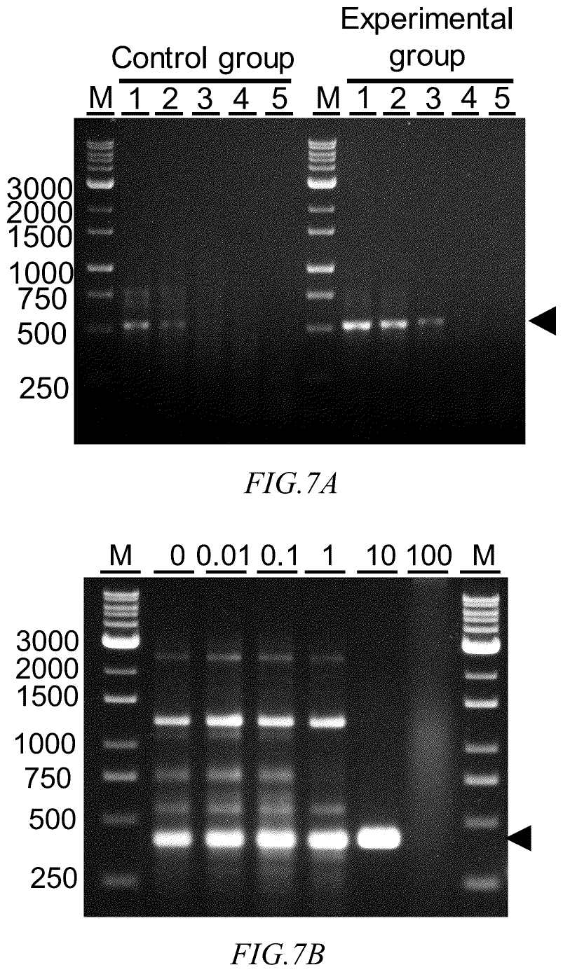

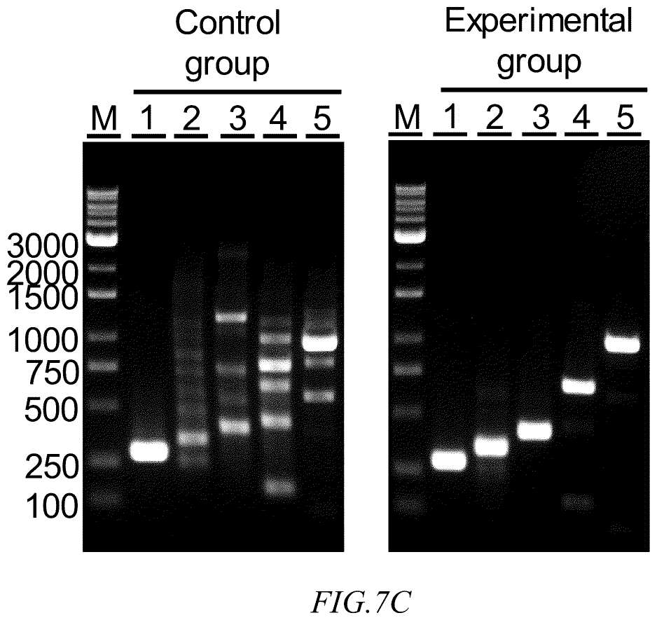

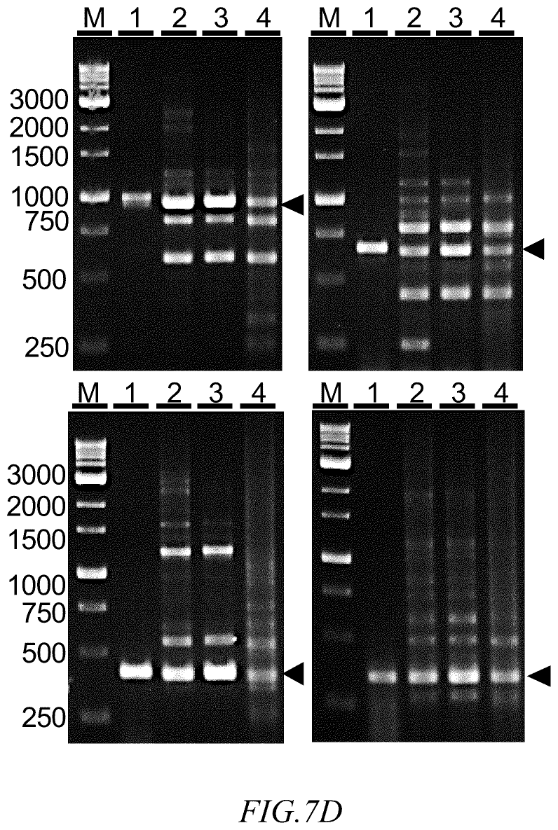

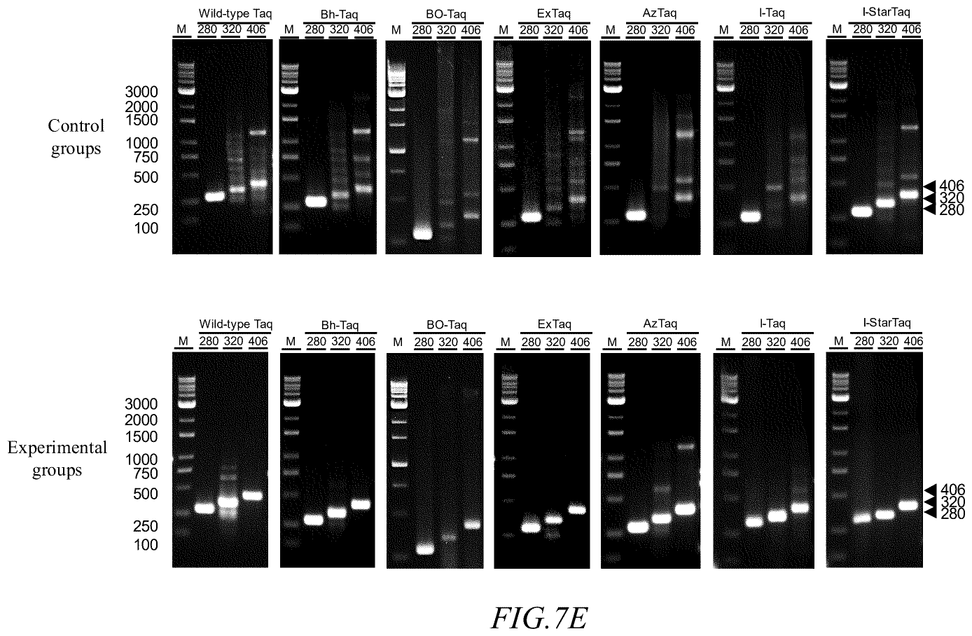

As shown in FIG. 7A, the control group exhibited weak or negligible amplification across the tested template inputs, whereas the group added with the saccharide nanomaterial demonstrated markedly improved amplification under the same conditions. These data indicate that the saccharide nanomaterial effectively enhances PCR amplification performance.

### 6.2 Concentration Test of Saccharide Nanomaterials

This experiment evaluated the effect of adding different concentrations of the saccharide nanomaterial to PCR on amplification performance.

### Materials and Methods

PCR was performed using 30 ng of a 406-bp human gDNA template with addition of 0 μg/mL (control), 0.01 μg/mL, 0.1 μg/mL, 1 μg/mL, 10 μg/mL, and 100 μg/mL of the sodium-alginate-derived nanomaterial, followed by gel electrophoresis. Unless otherwise indicated, PCR conditions were as set forth in Example 4.

### Results

As shown in FIG. 7B, the control group without the saccharide nanomaterial produced numerous nonspecific products. By contrast, groups supplemented with the saccharide nanomaterial exhibited fewer nonspecific products; in particular, addition of 1 g/mL resulted in a marked reduction of nonspecific products, and at 10 μg/mL the target amplicon was strongly expressed with disappearance of nonspecific products. These findings indicate that the saccharide nanomaterial effectively enhances PCR specificity.

### 6.3 Length Test of DNA Template

This experiment evaluated the effects of the saccharide nanomaterial and different template lengths on PCR amplification performance.

### Materials and Methods

PCR was performed using human gDNA templates of 280 bp (lane 1), 320 bp (lane 2), 406 bp (lane 3), 653 bp (lane 4), and 941 bp (lane 5). For each reaction, 30 ng of DNA template was used. The experimental group contained 10 μg/mL of the sodium-alginate-derived nanomaterial, and the control group contained no saccharide nanomaterial. The PCR products were subsequently analyzed by gel electrophoresis. Unless otherwise indicated, PCR conditions were as set forth in Example 4.

### Results

As shown in FIG. 7C, the control group exhibited numerous nonspecific bands after amplification, indicating poor specificity of the DNA polymerase in the absence of inhibition-commonly attributable to undesired low-temperature priming and extension at reaction initiation. In contrast, upon addition of the saccharide nanomaterial, nonspecific bands were not observed and the target-length amplicons were distinctly amplified. These findings demonstrate that inclusion of the saccharide nanomaterial effectively enhances PCR specificity and sensitivity, thereby substantially improving amplification accuracy.

### 6.4 Comparative Experiment of Commercially PCR Kits

This experiment compared the specificity and accuracy of PCR performed with the saccharide nanomaterial to those of commercially available hot-start PCR kits.

### Materials and Methods

PCR was performed using human gDNA templates of 320 bp, 406 bp, 653 bp, and 941 bp, with 30 ng of DNA template per reaction. Reactions supplemented with the saccharide nanomaterial (lane 1) were conducted in parallel with reactions using commercial hot-start PCR kits under the same conditions: DreamTaq DNA polymerase (Thermo Scientific, USA) (lane 2), DreamTaq Hot Start DNA polymerase (Thermo Scientific, USA) (lane 3), and KAPA2G Fast HotStart PCR Kit (Roche, Switzerland) (lane 4). The PCR products were subsequently analyzed by gel electrophoresis. Unless otherwise indicated, PCR conditions were as set forth in Example 4.

### Results

As shown in FIG. 7D, the commercial hot-start PCR kits (lanes 2-4) produced numerous nonspecific products. By contrast, the group supplemented with the saccharide nanomaterial (lane 1) exhibited virtually no nonspecific bands, demonstrating that the saccharide nanomaterial markedly enhances PCR specificity and performs superior to the tested commercial hot-start PCR kits.

### 6.5 Commercial PCR Polymerase Test

In this experiment, commercial PCR kits were supplemented with the saccharide nanomaterial described herein and subjected to PCR, followed by gel electrophoresis, to compare the effects of adding the saccharide nanomaterial on different DNA polymerases and on amplification performance.

### Materials and Methods

PCR was performed using human gDNA templates of 280 bp, 320 bp, and 406 bp, with 30 ng of DNA template per reaction. The control groups contained no saccharide nanomaterial, and the experimental groups contained 10 μg/mL of the saccharide nanomaterial. The PCR assay panels included: Wild-type Taq group (Wild Type Taq DNA Polymerase, Ten Giga Bio, Taiwan), Bh-Taq group (Taq DNA Polymerase, Bio-Helix, Taiwan), BO-Taq group (Gran Turismo PreMix, BiOptics, Taiwan), ExTaq group (Ex Taq DNA Polymerase, TaKaRa, Japan), AzTaq group (AZtaq DNA Polymerase, ArcticZymes, Norway), I-Taq group (I-Taq DNA Polymerase, LiliF Diagnostics, Korea), and I-StarTaq group (I-StarTaq DNA Polymerase, LiliF Diagnostics, Korea). Unless otherwise indicated, PCR conditions were as set forth in Example 4; reaction parameters for each commercial kit were adjusted according to the respective user manuals.

### Results

As shown in FIG. 7E, in the control groups without the saccharide nanomaterial, several commonly used commercial PCR kits (BO-Taq, ExTaq, AzTaq, and I-Taq) failed to yield complete amplification of the 320-bp and 406-bp targets (upper panel) and produced numerous nonspecific bands. Although the I-StarTaq group generated distinct target amplicons, it also exhibited substantial nonspecific bands. By contrast, upon addition of the saccharide nanomaterial to each PCR kit (experimental groups, lower panel), target amplicons that were previously not obtained were robustly produced and nonspecific amplification was markedly reduced. These results indicate that the saccharide nanomaterial is compatible with different DNA polymerases and, when incorporated into various PCR kits, effectively increases PCR specificity and sensitivity, thereby improving overall reaction accuracy.

### Example 7 Applications and Efficacy of PCR Testing

In this embodiment, multiplex detection of SARS-CoV-2 and its variant strain (Omicron) was performed to evaluate the effects of the saccharide nanomaterial on background interference and viral detection. In addition, polymerase chain reaction (PCR) employing primers for aldehyde dehydrogenase 2 (ALDH2) and its variant (E487K) was conducted to investigate the effect of the saccharide nanomaterial on primer-dimer formation and to assess its utility for single nucleotide polymorphism (SNP) detection.

### Materials and Methods

The saccharide nanomaterial was prepared as described in Example 1, wherein sodium alginate served as the saccharide precursor and the dry-heating temperature was 250° C.

Multiplex PCR for SARS-CoV-2 targeted three viral genes: RNA-dependent RNA polymerase (RdRp), envelope (E), and nucleocapsid (N). Distinct amplicon sizes were selected to enable resolution by capillary gel electrophoresis (CGE): 100 bp for RdRp, 113 bp for E, and 128 bp for N. Positive SARS-CoV-2 material was obtained from Ten Giga Bio (Taiwan). Each reaction contained 1,000 copies of viral plasmid genes, 10 ng of human genomic DNA, 500 nM of a primer mixture, and the saccharide nanomaterial, and was cycled for 45 cycles comprising denaturation at 95° C. for 15 s, annealing at 58° C. for 20 s, and extension at 72° C. for 30 s. Detection of the SARS-CoV-2 Omicron variant employed a two-step multiplex PCR workflow. Four separate reverse-transcription PCR (RT-PCR) reactions were performed, each including 30 ng of human genomic DNA, 5 ng of total human RNA, and 10,000 copies of synthetic SARS-CoV-2 Omicron RNA (BiOptic, Taiwan), with primer pairs directed to the Omicron envelope (E) gene. RT-PCR reagents (BiOptic, Taiwan) comprised 5×RT primers, 2×RT premix, and Rtase. Each 20 μL RT-PCR product was subsequently used as input for multiplex PCR. PCR with the saccharide nanomaterial comprised 44 cycles including denaturation at 95° C. for 15 s, annealing at 65° C. for 20 s, and extension at 72° C. for 20 s. Following PCR, the amplicons were mixed with 10- and 1,000-kb DNA markers and analyzed by CGE using an Si cartridge on a Qsep100 instrument (BiOptic, Taiwan). Signal-to-noise (S/N) for each target was quantified using Q-Analyzer software (BiOptic, Taiwan). Experimental groups contained 10 μg/mL of the sodium-alginate-derived nanomaterial; control groups were processed without the saccharide nanomaterial.

In further study, two sets of confronting primers were used to perform allele-specific direct PCR on human saliva specimens to detect the ALDH2 SNP variant, and the effect of adding the saccharide nanomaterial on primer-dimer formation was assessed. PCR cycling parameters and quantification were as described above.

### Results

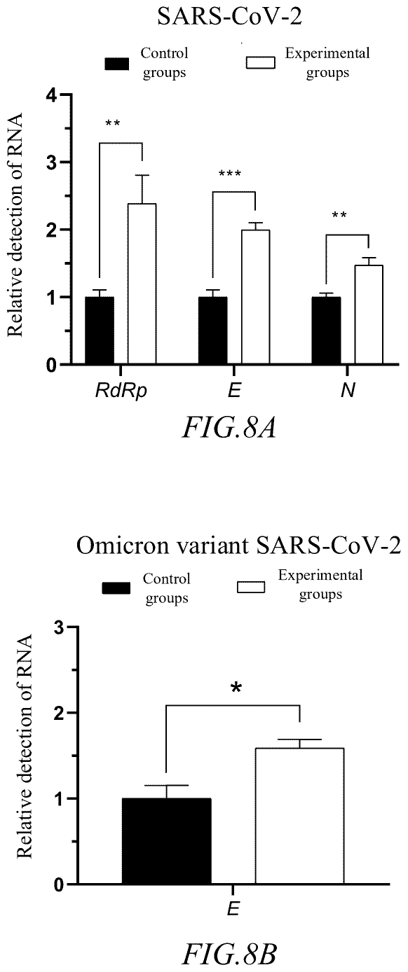

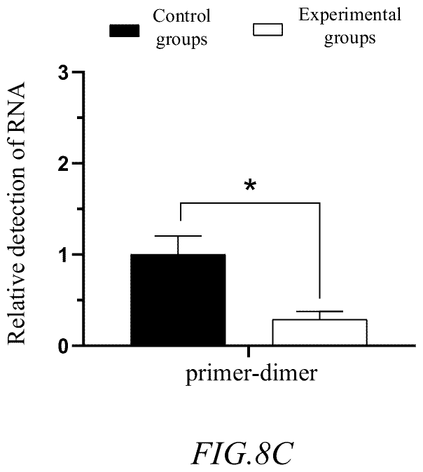

As shown in FIG. 8A, addition of the saccharide nanomaterial significantly enhanced detection signals for RdRp, E, and N by at least 1.5-fold, with RdRp exhibiting greater than a 2-fold increase. These findings indicate that, under complex multiplex conditions, the saccharide nanomaterial effectively improves PCR specificity and thereby overall diagnostic accuracy; by simultaneously targeting multiple regions of the viral genome, the approach mitigates false-positive and false-negative results. In addition, as shown in FIG. 8B, in detection of the SARS-CoV-2 Omicron variant, inclusion of the saccharide nanomaterial produced a marked increase in PCR signal. Collectively, the results demonstrate that the saccharide nanomaterial markedly improves amplification even in the presence of human cDNA background and is applicable to viral detection, further confirming that it enhances PCR specificity and facilitates more accurate identification of viruses and their variants.

As shown in FIG. 8C, in SNP detection of ALDH2, groups supplemented with the saccharide nanomaterial exhibited substantially fewer primer-dimer products, indicating that the saccharide nanomaterial is applicable to genetic disease testing and effectively improves PCR specificity by reducing primer-dimer formation.

### Example 8 Applications and Efficacy of qPCR Detection

In this embodiment, real-time PCR (qPCR) detection of enterovirus A71 (EV71) was performed to evaluate the effectiveness of the saccharide nanomaterial described herein in qPCR assays.

### Materials and Methods

The saccharide nanomaterial was prepared as described in Example 1, wherein sodium alginate served as the saccharide precursor and the dry-heating temperature was 250° C.

Quantitative reverse-transcription PCR (qRT-PCR) employed a viral nucleic acid extraction kit (BioHelix, Taiwan) following the manufacturer's instructions. RNA was extracted from human cell lines infected with EV71; plaque-forming units (PFU) were estimated by plaque assay. cDNA was synthesized using RT Premix (BiOptic, Taiwan). In this experiment, reactions containing RNA corresponding to 20, 200, 2,000, or 20,000 PFU, 1×RT primers, and reverse transcriptase (Rtase) were incubated at 55° C. for 15 min and then at 70° C. for 15 min. The experimental groups contained 10 μg/mL of the saccharide nanomaterial; the control groups contained no saccharide nanomaterial.

qPCR was performed with qPCR MasterMix (BioHelix, Taiwan) following the manufacturer's protocol, with a final reaction volume of 20 μL. Thermal cycling was conducted on an AriaMx real-time PCR system (Agilent, USA) with 95° C. initial denaturation for 5 min, followed by 40 amplification cycles of 95° C. for 30 s, 65° C. for 30 s, and 72° C. for 1 min, and a final extension at 72° C. for 5 min.

### Results

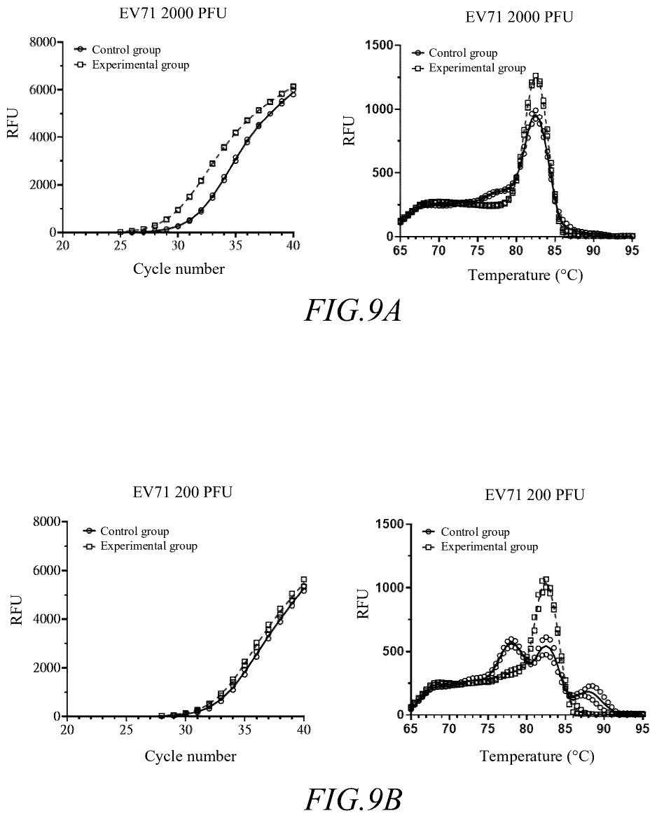

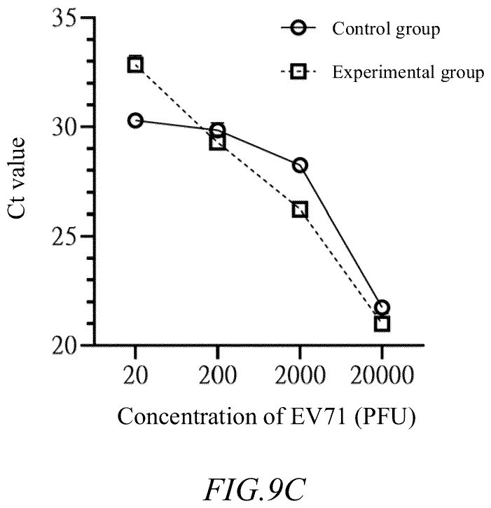

As shown in FIG. 9A, at a viral input of 2,000 PFU, the exponential amplification curves exhibited a higher relative fluorescence signal in the experimental group compared with the control, indicating that addition of the saccharide nanomaterial effectively enhances amplification efficiency.

As shown in FIG. 9B, at a viral input of 200 PFU, the experimental group likewise showed a higher relative fluorescence signal than the control, demonstrating improved amplification efficiency upon addition of the saccharide nanomaterial. Melting curve analysis further revealed that the control group (without saccharide nanomaterial) displayed two distinct peaks corresponding to the target amplicon and primer-dimer formation, indicating the presence of nonspecific signals arising from primer-dimers. In contrast, the experimental group supplemented with the saccharide nanomaterial effectively eliminated nonspecific signals and suppressed primer-dimer formation, thereby enabling more accurate determination of the cycle threshold (Ct).

As shown in FIG. 9C, the control group at 2,000 PFU exhibited a Ct value of 28.24, whereas the experimental group at 2,000 PFU showed a significantly lower Ct value of 26.23, indicating that the saccharide nanomaterial enhances qPCR amplification efficiency. In addition, the control group yielded similar Ct values at 20 PFU and 200 PFU, likely due to accumulation of nonspecific products during amplification, which compromised Ct accuracy. By contrast, in the experimental group, higher viral inputs produced lower Ct values with a clear linear relationship, further demonstrating that inclusion of the saccharide nanomaterial improves the reliability of qPCR detection.

In conclusion, the present invention provides saccharide nanomaterials prepared by dry heating of saccharide precursors. The saccharide nanomaterials can be generated from a variety of saccharides or saccharide derivatives. The surfaces of the saccharide nanomaterials present distinctive functional groups that effectively bind DNA polymerases and thereby inhibit polymerase activity. The inhibition is reversible: increasing temperature releases the polymerase from the saccharide nanomaterial without adversely affecting the enzyme's structure or catalytic activity. Leveraging this property, the saccharide nanomaterials are applied to hot-start polymerase chain reactions (PCR). Experimental data demonstrate that the saccharide nanomaterials markedly improve PCR specificity and sensitivity while substantially reducing nonspecific products and primer-dimer formation, thereby significantly enhancing assay accuracy. The saccharide nanomaterials are thus applicable to a variety of fields, including molecular biology workflows, viral detection, and genetic testing. By increasing reaction specificity and suppressing nonspecific amplification, more accurate quantitation can be achieved even at low target concentrations. Such enhanced specificity is critical for early detection of viral infections, enabling more timely and effective disease course management.

Moreover, relative to commercially available hot-start PCR kits, the saccharide nanomaterials provide more pronounced improvements in PCR specificity, increase amplification of the intended target, reduce formation of nonspecific products, and suppress primer-dimer formation, thereby mitigating false-positive and false-negative results. The saccharide nanomaterials are compatible with multiple classes of DNA polymerases and can be directly incorporated into existing PCR assay kits to immediately improve analytical accuracy. In addition, whereas conventional hot-start PCR often relies on costly antibodies or aptamers, the saccharide nanomaterials can be produced at low cost, are straightforward to prepare, and afford superior enhancement of PCR amplification performance.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A method for inhibiting the activity of DNA polymerase, comprising adding a saccharide nanomaterial to a DNA polymerase solution, wherein the saccharide nanomaterial is produced by dry heating a saccharide at 150° C. to 300° C., wherein the saccharide nanomaterial comprises graphene-like nanosheet and the saccharide, wherein the graphene-like nanosheet comprise a carbonized product of at least a portion of the saccharide, and the graphene-like nanosheet is complexed with the saccharide to form a cross-linked supramolecular structure.

2. The method of claim 1, wherein the saccharide is selected from the group consisting of monosaccharide, disaccharide, oligosaccharide, polysaccharide and any combination thereof.

3. The method of claim 1, wherein the saccharide is selected from the group consisting of glucose, maltose, dextrin, sodium alginate, agarose, hydroxyethyl cellulose, and xanthan gum.

4. The method of claim 1, wherein the cross-linked supramolecular structure further has a functional group selected from the group consisting of hydroxyl, ester, phenol, carboxyl and any combination thereof on a surface thereof.

5. The method of claim 1, wherein the saccharide nanomaterial inhibits the activity of the DNA polymerase by binding to the DNA polymerase, and the inhibition is reversible.

6. The method of claim 1, wherein the method is used in a hot-start polymerase chain reaction, wherein the saccharide nanomaterial binds to the DNA polymerase to inhibit its activity, and the DNA polymerase is released from the saccharide nanomaterial upon an increase in reaction temperature, thereby restoring the activity of DNA polymerase.

7. A method for improving the accuracy of polymerase chain reactions, comprising adding a saccharide nanomaterial to the polymerase chain reaction, wherein the saccharide nanomaterial is produced by dry heating a saccharide at 150° C. to 300° C., wherein the saccharide nanomaterial comprises graphene-like nanosheet and the saccharide, wherein the graphene-like nanosheet comprise a carbonized product of at least a portion of the saccharide, and the graphene-like nanosheet is complexed with the saccharide to form a cross-linked supramolecular structure.

8. The method of claim 7, wherein the saccharide nanomaterial binds to the DNA polymerase to inhibit its activity, and the DNA polymerase is released from the saccharide nanomaterial upon an increase in reaction temperature, thereby restoring the activity of DNA polymerase.

9. The method of claim 7, wherein the concentration of the saccharide nanomaterial is 1 μg/ml to 2500 μg/ml.

10. The method of claim 7, wherein the polymerase chain reaction comprises conventional polymerase chain reaction (PCR), hot-start polymerase chain reaction (hot-start PCR), capillary electrophoresis polymerase chain reaction (PCR-capillary electrophoresis), real-time polymerase chain reaction (real-time PCR), multiplex polymerase chain reaction (multiplex PCR), or allele-specific polymerase chain reaction (allele-specific PCR).
